(19) United States
(12) Patent Application Publication
(10) Pub. No.: US 2012/0100395 A1
(43) Pub. Date: Apr. 26, 2012

(54) INDICATOR SYSTEM FOR MONITORING A STERILIZATION PROCESS

(75) Inventors: Leonhard Feiler, Binzen (DE); Hans Reichert, Rheinfelden (DE); Véronique Hall-Goulle, Dornach (CH); Thomas Raimann, Sisseln (CH)

(73) Assignee: BASF SE, Ludwigshafen (DE)

(21) Appl. No.: 13/319,460

(22) PCT Filed: May 5, 2010
(86) PCT No.: PCT/EP2010/056065

§ 371 Date: Jan. 12, 2012

(30) Foreign Application Priority Data
May 8, 2009 (EP) 09159800.3

(51) Int. Cl. A61L 2/04; B32B 9/04; G01N 31/22; G01N 25/00

(52) U.S. Cl. 428/704; 252/408.1; 422/22; 116/207

(57) ABSTRACT

The present invention relates to a device for monitoring integral value of time, temperature comprising at least one layer of polymer comprising a latent pigment capable of undergoing at least one colour change, and an organic acid; or comprising one layer of polymer comprising a latent pigment capable of undergoing at least one colour change, and one layer of polymer comprising an organic acid, wherein said latent pigment is converted to its pigmentary form which causes said colour change. The device can be used for monitoring sterilization of medical and kitchen supplies, canned foods and doneness of microwave foods.

The present invention relates to a colour-changing device for monitoring integral value of time and temperature. The device can be used for monitoring sterilization of medical and kitchen supplies, canned foods and doneness of microwave foods.

A wide variety of medical supplies are sterilized with materials and techniques, such as steam, ethylene oxide, plasma, peracetic acid, formaldehyde and high-energy radiation. Kitchenware, such as dishes, cutlery, and utensils used at home and restaurants are also sterilized in dishwashers with hot water and hot air usually around 90° C. It is essential to assure that these items are sterilized.

In order to assure the sterilization with heat, the indicator, or dosimeter, must determine integral value of two parameters viz. time and temperature. It is often desirable that the indicator is essentially unaffected by other parameters, humidity, steam, ethylene oxide and radiation.

Pre-cooked frozen food is widely used today. The pre-cooked frozen food is heated either in a conventional oven (for example, heated with natural gas or electricity) or more conveniently in a microwave oven. A microwave oven does not heat the food uniformly. Some portions of food may not be done while the other portions may be over heated.

Homes, restaurants and catering organizations use kitchenware such as dishes, cutlery and utensils, which need to be sterilized with either dry heat, hot water and steam usually below 100° C. There is also a need for an indicator to make sure that the cookware has been subjected to certain integral value of heat.

A wide variety of foods especially canned foods, pharmaceuticals, hospital and medical supplies are sterilized. These and other products such as linens are sterilized to kill living organisms to an acceptable level. Direct testing for sterility is destructive and expensive and hence indirect testing methods, such as color changing indicators are used.

Many steam sterilization indicators are reported in the literature and some of them are used for monitoring sterilization. A few of them use heavy and toxic metals, such as lead. There is a need for a sterilization indicator that does not use toxic and heavy metals.

U.S. Pat. No. 3,523,011 describes an indicator material consisting of calcium sulfide and lead carbonate. Upon exposure to steam at 120° C., calcium sulfide decomposes to form calcium hydroxide and hydrogen sulfide. The hydrogen sulfide reacts with lead carbonate to form black lead sulfide. Steam sensitive composition of U.S. Pat. No. 5,064,576 contains a metal complex (e. g. zirconium chloranilate) and an exchange ligand (e. g. citric or tartaric acid salts and amino carboxylic acid), binder (e. g. nitrocellulose and ethylcellulose) and a color change rate regulator (e. g. Resino blue, Resino yellow). U.S. Pat. No. 4,514,361 discloses a steam sterilization indicator containing a carrier (e. g. filter paper), a pH value indicator (e. g. bromocresol purple) and a chemical composition that contains (a) 2,4-dihydroxybenzoic acid and its metal salt and (b) phenylpropionic acid and its metal salt. Under steam sterilization conditions, the pH of mixture exceeds predetermined pH (5.8 to 6.2) due to formation of carbonate or bicarbonate (basic), causing the indicator to change color and indicate that sterilization is complete. U.S. Pat. No. 5,158,363 describes a steam sterilization indicator, which contain (a) water-soluble organic compound whose melting point in the absence of steam is greater than sterilization temperature and (b) ink dye. Upon steam exposure, dye changes colour from clear to dark brown or black. U.S. Pat. No. 5,087,659 describes ink composition as steam sterilization indicators for use in jet printing. The composition uses an organic dyestuff, which forms a salt with phenol resin. The ink composition is discoloured or changes colour under steam sterilization conditions. U.S. Pat. No. 3,981,683, U.S. Pat. No. 3,932,134, U.S. Pat. No. 4,195,055 and U.S. Pat. No. 4,410,493 illustrate processes, which use permeation or wicking of an indicator chemical (such as sebasic acid and salicylamide) and a dye. A disposable pre-vacuum steam sterilizer test device is described in U.S. Pat. No. 4,486,387. Other indicators for noting the completion of steam sterilization are reported in U.S. Pat. Nos. 4,121,714; 3,360,339; 2,826,073; 3,568,627; 3,360,338; 2,798,885 ; 3,386,807; 3,360,337; and 3,862,824. The indicators, which monitor integral value of time, temperature and humidity are often commonly referred to as steam indicators herein.

WO01/10471 discloses ink formulations and devices for monitoring sterilization with ethylene oxide. The device is made by coating a mixture of (a) a polymeric binder, (b) a ethylene oxide reactive salt, such as sodium thiocyanate and tetraethylammonium bromide and (c) a pH sensitive dye, such as bromothymol blue and bromocresol purple. When contacted with ethylene oxide, the device undergoes at least one color change due to production of a base such as sodium hydroxide. However, these devices and formulations are selective to ethylene oxide only.

WO00/61200 discloses formulations and devices for monitoring sterilization with plasma. The device is made by coating of a mixture of at least one (a) polymeric binder, (b) plasma activator and (c) plasma indicator. The device undergoes a color change when treated with plasma, especially that of hydrogen peroxide. For example, when a coating of phenol red and tetraethylammonium bromide in a binder, such as polyacrylate undergoes a color change from yellow-to-blue when exposed to hydrogen peroxide and its plasma due to halogenation of the dye. However, these devices and formulations are selective to plasma only.

WO9633242A2 relates to indicator ink compositions that contains a water-based dispersion of a phenol-formaldehyde resin, at least one colorant and an amine-terminated catalyst. These indicator ink compositions are fixed to a surface, such as by drying, and respond to specific conditions of time, temperature, humidity, pressure and the presence or absence of certain chemicals by changing color. Compositions provide a means for detecting exposure to a predetermined condition and can be fixed or otherwise attached to nearly any article or designed into patterns on materials. Exposure of the indicator ink to the specific condition, the indicator ink provides a permanent detectable record of the event. Further, by varying the amount of amine-terminated catalyst in the formulation, the specific conditions at which the indicator composition will change colour can be altered. Such compositions are specifically useful for determining the effectiveness of a sterilization process.

WO0061200A1 relates to a device for monitoring sterilization with plasma comprising at least one layer of polymer, having incorporated therein an indicator capable of undergoing at least one colour change, an activator for the indicator. Wherein said activator is contacted with said plasma, it undergoes a reaction with it to produce a product which causes the indicator to undergo a colour change. There is further provided a process for making this device and for using it.

EP1990064A1 describes a sterilization test pack, comprising a first chamber, a source of active enzymes within the first chamber, a lumen in fluid communication with the first chamber and the source of active enzymes within the chamber, and a second chamber containing an indicator reagent mixture, the second chamber in fluid communication with the first chamber so that the indicator reagent mixture may be brought into contact with the source of active enzymes after a sterilization cycle is complete.

WO2009023503A1 relates to an indicator composition comprising at least one iodide salt, at least one binder, at least one carbonate salt and/or at least one sulfate salt, and at least one antioxidant. The indicator composition may contain at least one dye, complexing agent, inhibitor, and/or solvent. A sterilization indicator comprising the foregoing indicator composition is disclosed. The sterilization indicator may be used for monitoring sterilization processes involving oxidative chemistries.

WO2008082728A2 relates to a sterilization indicator and a process to concentrate signal generated by constraining it to a minimal surface, in a minimal volume and minimal pH and growth buffering or mediating influences. The sterilization indicator may comprise a carrier, the carrier having a first surface and a second surface; a support, the support having a first section and a second section, the carrier overlying the first section of the support, the second surface of the carrier being adhered to the first section of the support; and a biological indicator supported by the carrier. The second section of the support may be of sufficient dimension to permit handling the sterilization indicator without contacting the biological indicator. A process for making the sterilization indicator and processes for using the sterilization indicator are disclosed.

WO0186289 discloses a device for monitoring integral value of time, temperature and water vapor comprising at least one layer of polymer comprising an isomeric indicator capable of undergoing at least one color change, a controller for said indicator capable of influencing the time and temperature required for said color change when contacted with water vapor, wherein said indicator undergoes an isomerization reaction which causes said indicator to undergo said color change.

WO2008008208A2 relates to a multilayer food sterilization dosage indicator having (a) an indicator layer including an ion-sensitive ink, an ionic photoinitiator, a proton source; (b) a substrate, and an overlayer including a UV-absorber. The ink undergoes a color change upon exposure to a known amount of sterilizing radiation.

DE10303914A1 discloses formulations containing colorant comprising one, or more irreversibly thermochromic latent pigments undergoing a significant color change below 200° C. The formulations are used to form printed products. By means of IR laser the colour of the printed products can be changed. For example, barcodes can be generated, which are machine readable.

According to JP63274584 a coloring matter or its precursor and an acid material are dissolved in an organic solvent, wherein vapor pressure of 30 wt. % or more of the total organic solvent amount is at most 50 mmHg at 20° C. and the viscosity is 5 cp or less at 20° C. In this manner, a composition for identification of a dosage of a desensitizing ink for pressure-reduction paper is prepared. Alizarin Red, Bromocresol Green, etc. are used as the coloring matter or its precursor, and benzoic acid, salicylic acid, lauric acid, citric acid, etc. are used as acid materials.

US2008145948 provides a chemical indicator test strip, comprising: a substrate; and a coating comprising a leuco dye complex on the substrate, the coating being insoluble in water and reactive with an organic analyte, the coating derived from a solution of leuco dye and developing agent. In another aspect, the coating further comprises adjuvant, in the form of a water-insoluble, polar, hydrophobic, aprotic material that extends the lower limit of detection for the analyte.

U.S. Pat. No. 4,407,960 relates to an indicator system and device for visually monitoring ethylene oxide sterilization. The indicating composition comprises a leuco precursor of an aryl methane dye selected from the groups herein defined; and an acidic constituent. Acidic organic compounds such as diphenolic acid (4,4-bis[4-hydroxyphenyl]pentanoic acid) are effective acid constituents because they enhance hue, develop color and stabilize the final color change.

U.S. Pat. No. 5,840,449 provides a process for the preparation of a material comprising a substrate, comprising (a) coating the substrate with a solution or melt of at least one latent pigment and (b) converting the latent pigment partially or completely into its insoluble pigment form. U.S. Pat. No. 5,840,449 also relates to a structured material prepared in this way, to its use as a color filter or for the permanent storage of digital information, and to a method of reading out digital information stored thereon by irradiation with a light source and measurement of the intensity of the reflected or transmitted light beam. According to U.S. Pat. No. 5,840,449 the conversion (b) of the latent pigments into their insoluble form can be carried out very easily by, alternatively, thermal treatment (heating at temperatures for example between 50 and 400° C., preferably at from 100 to 200° C., especially by the close action of a heat source or by irradiation with infrared light), photolytic treatment (irradiation, for example, with UV light of wavelength .ltoreq.375 nm) or chemical treatment (exposure to the vapour of an organic or inorganic Bronsted or Lewis acid or of a base, such as hydrochloric acid, trifluoroacetic acid, boron trifluoride, ammonia or 4-dimethylaminopyridine).

U.S. Pat. No. 6,495,250 relates to a colored, preferably natural porous material comprising in its pores an effectively coloring amount of an organic pigment which is obtained by fragmenting a meltable or solvent-soluble pigment precursor in the presence of a thermally activated acid precursor characterized in said fragmentation takes place in the presence of an effective amount of a strong acid obtained from a catalyst precursor at a temperature of from 40 to 160° C., and to a low to medium temperature process for its preparation. The strong acid resulting from the catalyst precursor's thermal reaction has preferably a pKa, of 2 or lower, most preferably a pKa of 1 or lower.

US2007269737 relates to compositions and methods for forming color images on a substrate capable of development at desired wavelengths or energy levels. The color forming composition can include a carrier system and a latent pigment with a pendent group, wherein the latent pigment is colorless or pale in color while the pendent group is attached, and wherein the latent pigment is convertible to a colored pigment upon removal of the pendent group.

In Example 2 of US2007269737 a UV-curable matrix having a p-toluene sulfonic acid (pkₐ=0.7) and a radiation antenna dye dissolved therein is prepared. The latent pigment used is Latent Pigment 3,

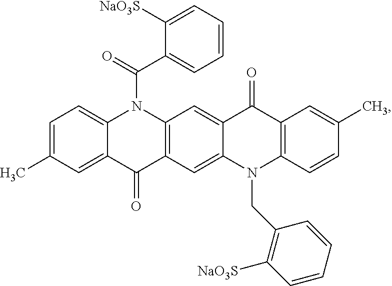

which is insoluble in the UV curable matrix. Exposing a 1-10 μm thick coating of the composition prepared above to a wavelength matched to the radiation antenna dye (generating heat of at least 130° C.) causes melting and/or diffusion of Latent Pigment 3. Upon interaction between the p-toluene sulfonic acid and Latent Pigment 3, magenta pigment PR122 is generated:

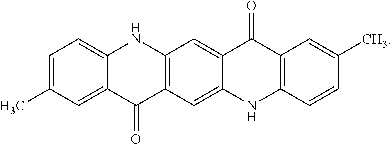

It is an object of the present invention to provide an indicator which can monitor integral value of temperature and time. It is another object of the present invention to provide an indicator which is economical to manufacture and use.

Provided is a device for monitoring integral value of time and temperature comprising at least one layer of polymer comprising a latent pigment capable of undergoing at least one colour change, and an organic acid; or comprising one layer of polymer comprising a latent pigment capable of undergoing at least one colour change, and one layer of polymer comprising an organic acid (which can also be a photoacid generator (PAG) or a thermal acid generator (TAG)), wherein said latent pigment is converted to its pigmentary form which causes said colour change; as well as
a process of making the device, comprising: a) applying the indicator formulation of the present invention to a substrate;
a process of using the device for monitoring sterilization of materials comprising the steps of: a) affixing said device to said materials or containers containing said materials; b) carrying out a process of sterilization; and d) observing a colour change indicating said sterilization has proceeded. The latent pigment is a pigment comprising groups, which makes the pigment soluble in the application medium and which can be split off by heat, or radiation, whereby the latent pigment is converted to the pigment form and the organic acid has a pKₐ-value in the range of from 1 to 5.

Such a device can be used for monitoring sterilization of medical supplies and canned foods, and doneness of microwave foods.

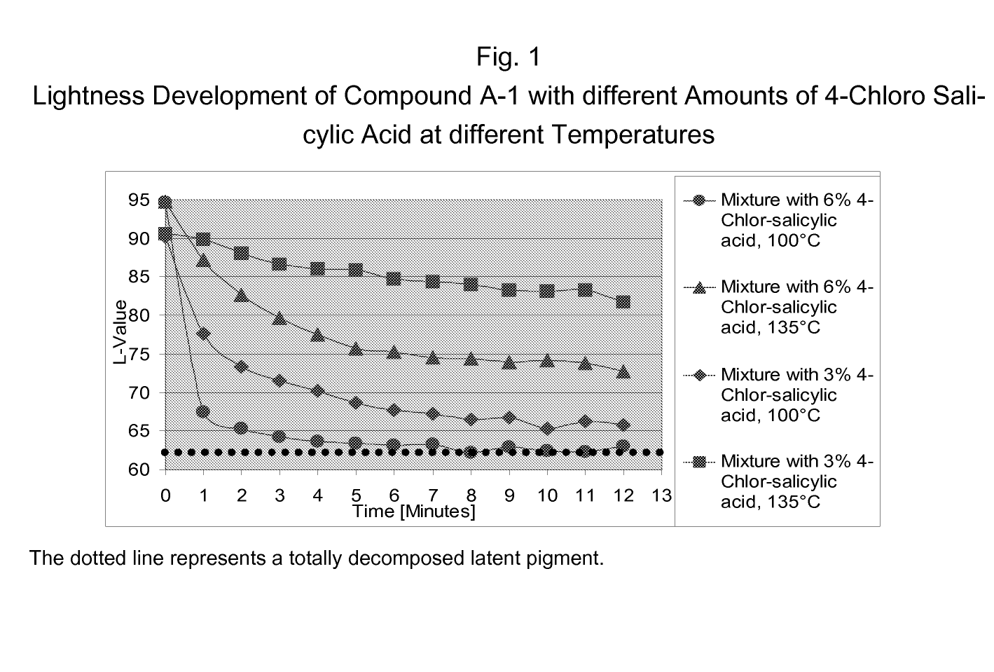

FIG. 1 shows the lightness development of compound

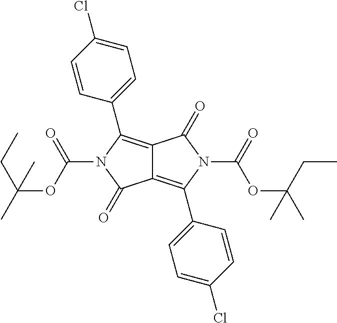

(A-1) with different amounts of 4-chloro salicylic acid (B-2) at different temperatures.

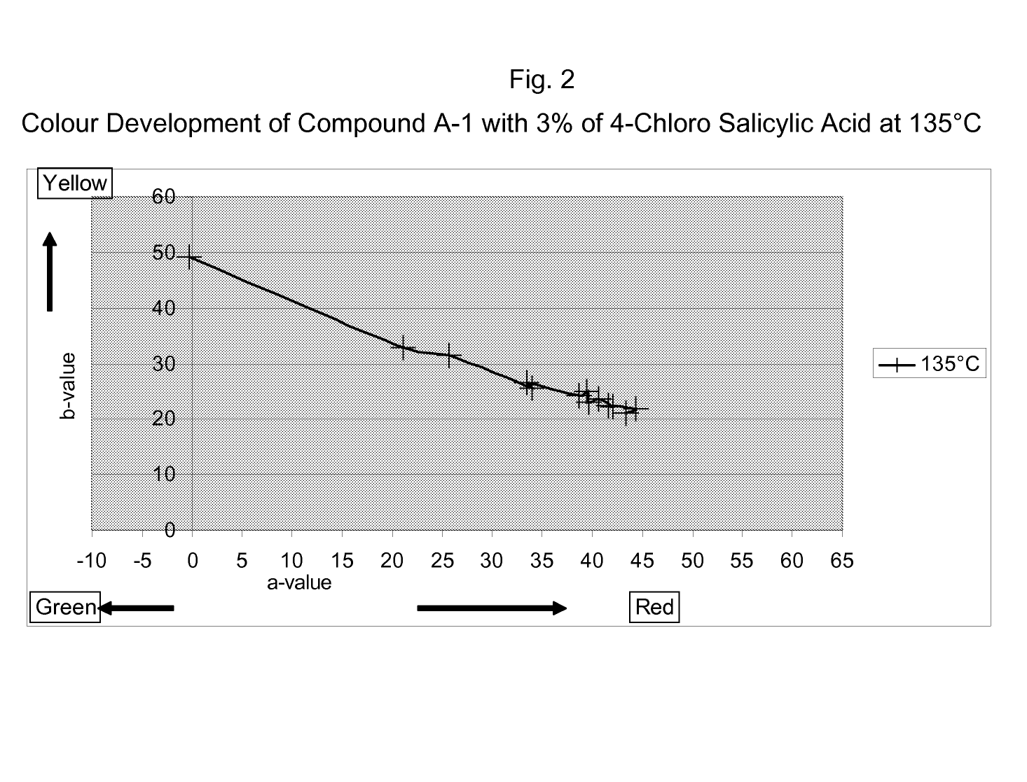

FIG. 2 shows the colour development of compound A-1 with 3% of 4-chloro salicylic acid (B-2) at 135° C.

The device could have more than one indicator layers each containing indicator, organic acid and binders. Both layers do not have to undergo colour changes. The indicator can have an optional topcoat or can be laminated with a transparent film. The indicator can also be sandwiched between two layers, one preferably clear for viewing color change. The indicator can also be composed of two layers one of which contains the latent pigment and one of which contains the organic acid.

Desired colours and colour changes can be obtained by mixing proper latent pigments optionally with other coloring agents in appropriate amounts. Similarly, the time required for the colour change can be varied by using a proper mixture of the latent pigments and organic acids in appropriate amounts. The desired colours and the time required for the colour changes can be obtained by selecting a proper mixture of compatible binders, and additives.

Though the device could be a self-supporting polymer film containing the latent pigment and organic acid, it is desirable to prepare the device on a substrate. The device can be made by coating the indicating formulation on a substrate. The substrate could be any solid surface, for example, that made from one or more polymeric materials, paper, ceramics, metals, woven fibers, non-woven fibers, or a combination of two or more thereof.

The substrate could be a container, e. g., bag, pouch, can or container lid, for items to be sterilized or food to be cooked. The sterilization indicator can also be prepared in form of stickers, strips and tapes.

Although any solid substrate having a smooth surface can be used, a preferred substrate is a flexible and transparent plastic film, and natural (cellulose) and synthetic (e. g., spun bonded polyolefins, e. g., Tyvak®) papers. Plastic films, such as polyethylene, polypropylene, polyvinyl chloride, polymethylmethacrylate, polyurethanes, nylons, polyesters, polycarbonates, polyvinyl acetate, cellophane and esters of cellulose can be used as the transparent substrate. Metal foils, such as aluminum can be used. The most preferred substrates are the 5-300 microns thick films of polyethylene terephthalate, cellulose paper and Tyvak®. The indicator could be in the form of any shape, e. g., dot, square, rectangle, picture, image and message.

The device is made by a) applying the indicator formulation of the present invention to a substrate.

The device can also contain a reference colour to help the user to determine the finalisation of the sterilisation process. The colour of the reference colour is adjusted to the colour of the indicator layer after a predefined time-temperature integral.

The device offers many advantages including: the formulations are inexpensive; the ingredients are considered nontoxic; it is easy to make the ink formulations, just by mixing proper ingredients in an ink extender; the ink has required pot life; there is no bleeding/diffusion of dyes; ink is printable with gravure and flexo presses on polyester, paper and type; the time required for the colour change can be varied by simple means; and it provides desired colour changes (from a starting light colour, such as yellow to a final dark colour, such as bordeaux red).

The (sterilisation) indicator formulation comprises a latent pigment, an organic acid having a pKₐ-value in the range of from 1 to 5, and a binder.

The so-called “latent pigment” is a pigment comprising groups, which makes the pigment soluble in the application medium and which can be split off by heat, or radiation, whereby the latent pigment is converted to the pigment form.

The latent pigment generally has the following formula A(B)ₓ (I) wherein

x is an integer from 1 to 8,

A is the radical of a chromophore of the quinacridone, anthraquinone, perylene, indigo, quinophthalone, indanthrone, isoindolinone, isoindoline, dioxazine, azo, phthalocyanine or diketopyrrolopyrrole series, which is linked to x groups B by one or more hetero atoms, those hetero atoms being selected from the group consisting of nitrogen, oxygen and sulfur and forming part of the radical A,

B is a group of the formula

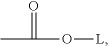

it being possible for the groups B, when x is a number from 2 to 8, to be the same or different, and

L is any desired group suitable for imparting solubility.

L is preferably a group of formula

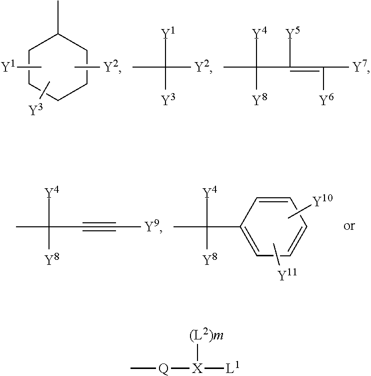

wherein Y¹, Y² and Y³ are each independently of the others C₁-C₆alkyl,

Y⁴ and Y⁸ are each independently of the other C₁-C₆alkyl, C₁-C₆alkyl interrupted by oxygen, sulfur or N(Y¹²)₂, or unsubstituted or C₁-C₆alkyl-, C₁-C₆alkoxy-, halo-, cyano- or nitro-substituted phenyl or biphenyl,

Y⁵, Y⁶ and Y⁷ are each independently of the others hydrogen or C₁-C₆alkyl,

Y⁹ is hydrogen, C₁-C₆alkyl or a group of formula

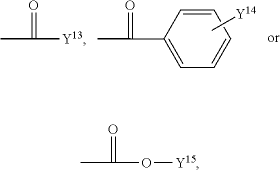

Y¹⁰ and Y¹¹ are each independently of the other hydrogen, C₁-C₆alkyl, C₁-C₆alkoxy, halogen, cyano, nitro, N(Y¹²)₂, or unsubstituted or halo-, cyano-, nitro-, C₁-C₆alkyl- or C₁-C₆alkoxy-substituted phenyl,

Y¹² and Y¹³ are C₁-C₆alkyl, Y¹⁴ is hydrogen or C₁-C₆alkyl, and Y¹⁵ is hydrogen, C₁-C₆alkyl, or unsubstituted or C₁-C₆alkyl-substituted phenyl,

Q is p,q-C₂-C₆alkylene unsubstituted or mono- or poly-substituted by C₁-C₆alkoxy,

C₁-C₆alkylthio or C₂-C₁₂dialkylamino, wherein p and q are different position numbers,

X is a hetero atom selected from the group consisting of nitrogen, oxygen and sulfur, m being the number 0 when X is oxygen or sulfur and m being the number 1 when X is nitrogen, and

L¹ and L² are each independently of the other unsubstituted or mono- or poly-C₁-C₁₂alkoxy-, —C₁-C₁₂alkylthio-, —C₂-C₂₄dialkylamino-, —C₆-C₁₂aryloxy-, —C₆-C₁₂arylthio-, —C₇-C₂₄alkylarylamino- or —C₁₂-C₂₄diarylamino-substituted C₁-C₆alkyl or [-(p′,q′-C₂-C₆alkylene)-Z—]ₙ—C₆alkyl, n being a number from 1 to 1000, p′ and q′ being different position numbers, each Z independently of any others being a hetero atom oxygen, sulfur or C₁-C₆alkyl-substituted nitrogen, and it being possible for C₂-C₆alkylene in the repeating [—C₂-C₆alkylene-Z—] units to be the same or different,

and L1 and L2 may be saturated or unsaturated from once to ten times, may be uninterrupted or interrupted at any location by from 1 to 10 groups selected from the group consisting of —(C═O)— and —C₆H₄—, and may carry no further substituents or from 1 to 10 further substituents selected from the group consisting of halogen, cyano and nitro. Of special interest are compounds of formula (I) wherein L is

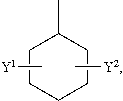

C₁-C₆alkyl, C₂-C₆alkenyl or

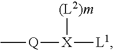

wherein Q is C₂-C₄alkylene, Y¹ and Y² are each independently of the others C₁-C₆alkyl, and L¹ and L² are [—C₂-C₁₂alkylene-Z—]ₙ—C₁-C₁₂alkyl or is C₁-C₁₂alkyl mono- or poly-substituted by C₁-C₁₂alkoxy, C₁-C₁₂alkylthio or

C₂-C₂₄dialkylamino, and m and n are as defined hereinbefore.

Of very special interest are compounds of formula (I) wherein L is

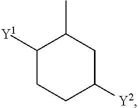

C₄-C₅alkyl, C₃-C₆alkenyl or

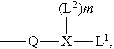

wherein Q is C₂-C₄alkylene, Y¹ and Y² are each independently of the others C₁-C₆alkyl, X is oxygen and m is zero, and L¹ is [—C₂-C₁₂alkylene-O—]ₙ—C₁-C₁₂alkyl or is C₁-C₁₂alkyl mono- or poly-substituted by C₁-C₁₂-alkoxy, especially those wherein -Q-X— is a group of formula —C(CH₃)₂—CH₂—O—.

Examples of suitable compounds of formula (I) are disclosed in EP-A-0 648 770, EP-A-0 648 817, EP-A-0 742 255, EP-A-0 761 772, WO98/32802, WO98/45757, WO98/58027, WO99/01511, WO00/17275, WO00/39221, WO00/63297 and EP-A-1 086 984. The pigment precursors may be used singly or also in mixtures with other pigment precursors or with colorants, for example customary dyes for the application in question.

A is the radical of known chromophores having the basic structure A(H)ₓ, wherein A preferably has, at each hetero atom linked to x groups B, at least one immediately adjacent or conjugated carbonyl group, such as, for example,

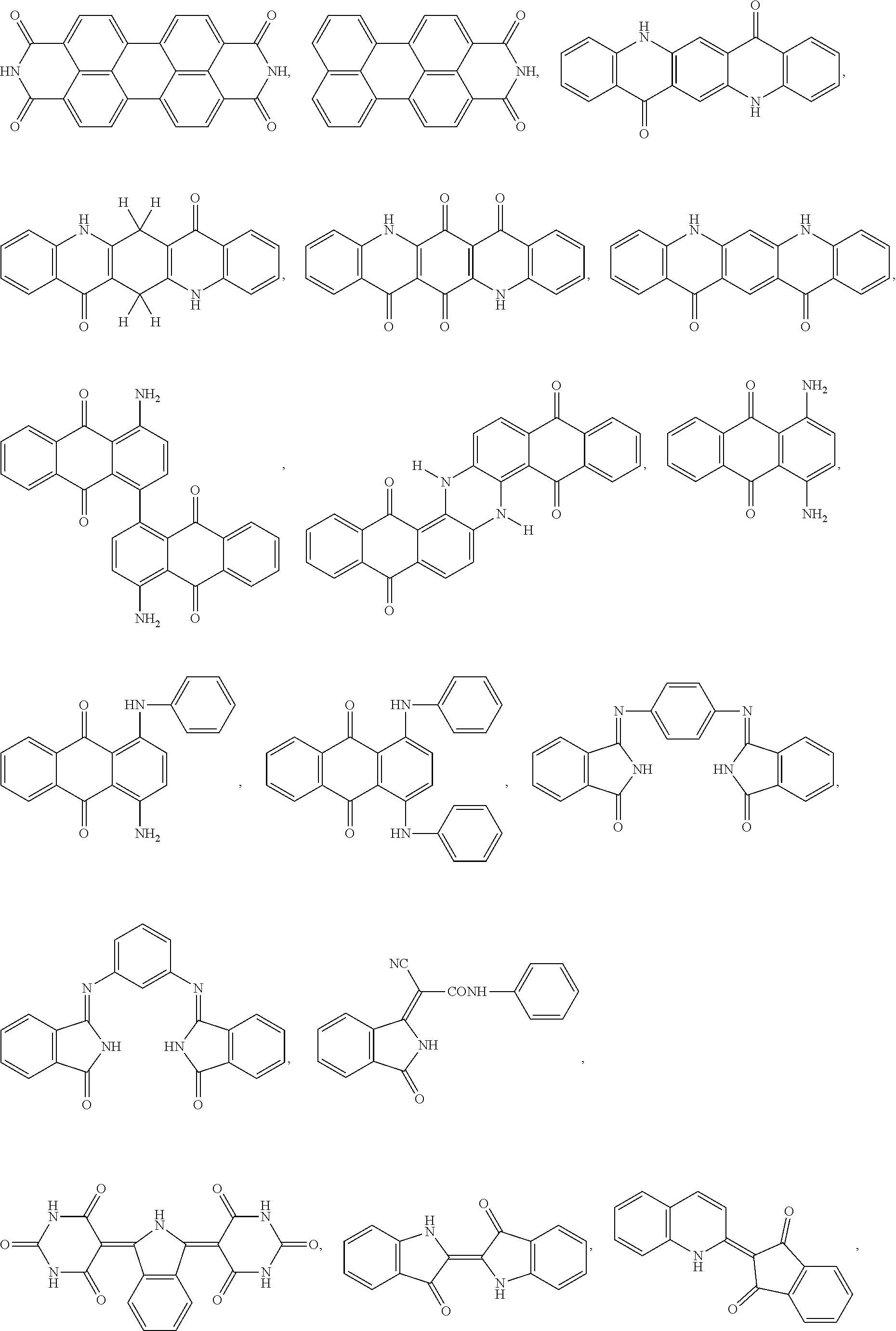

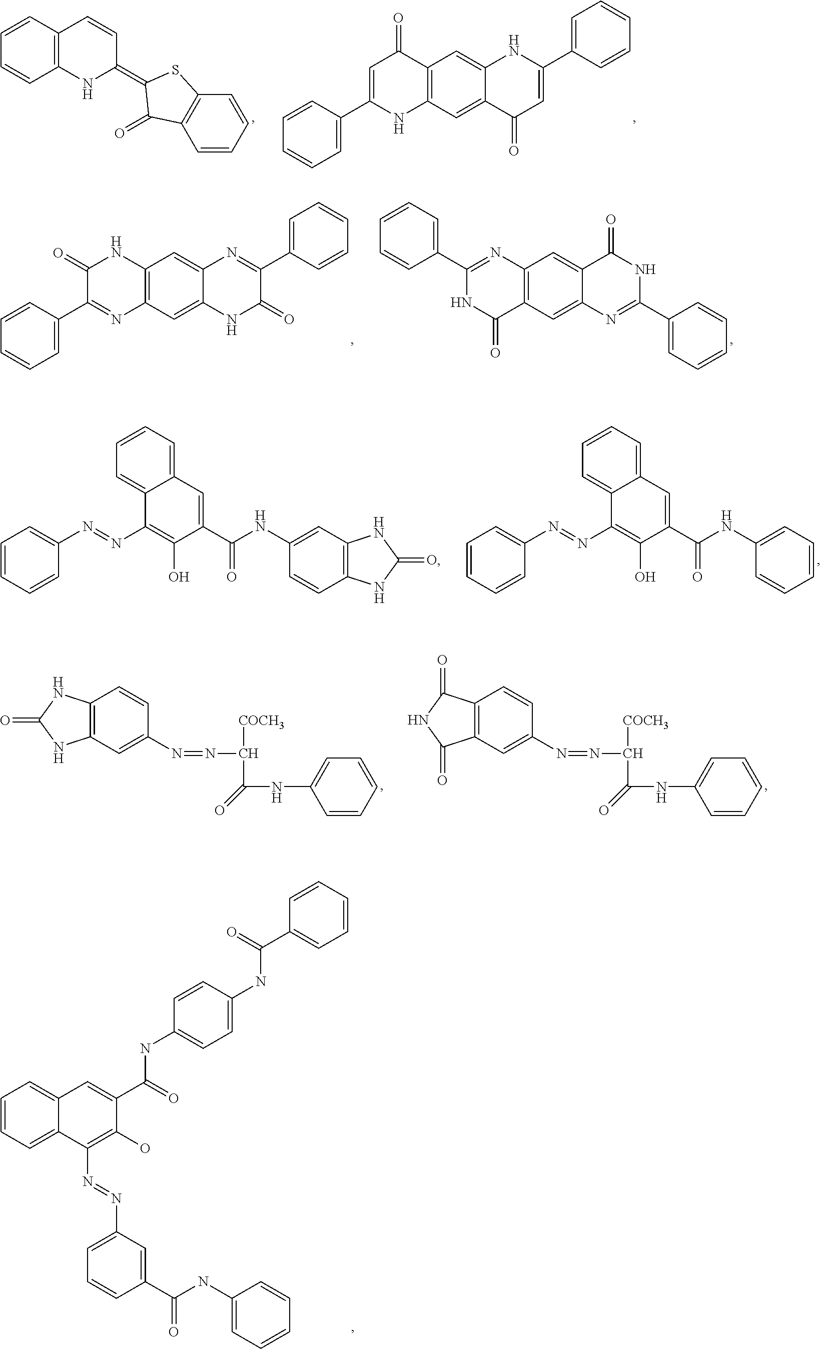

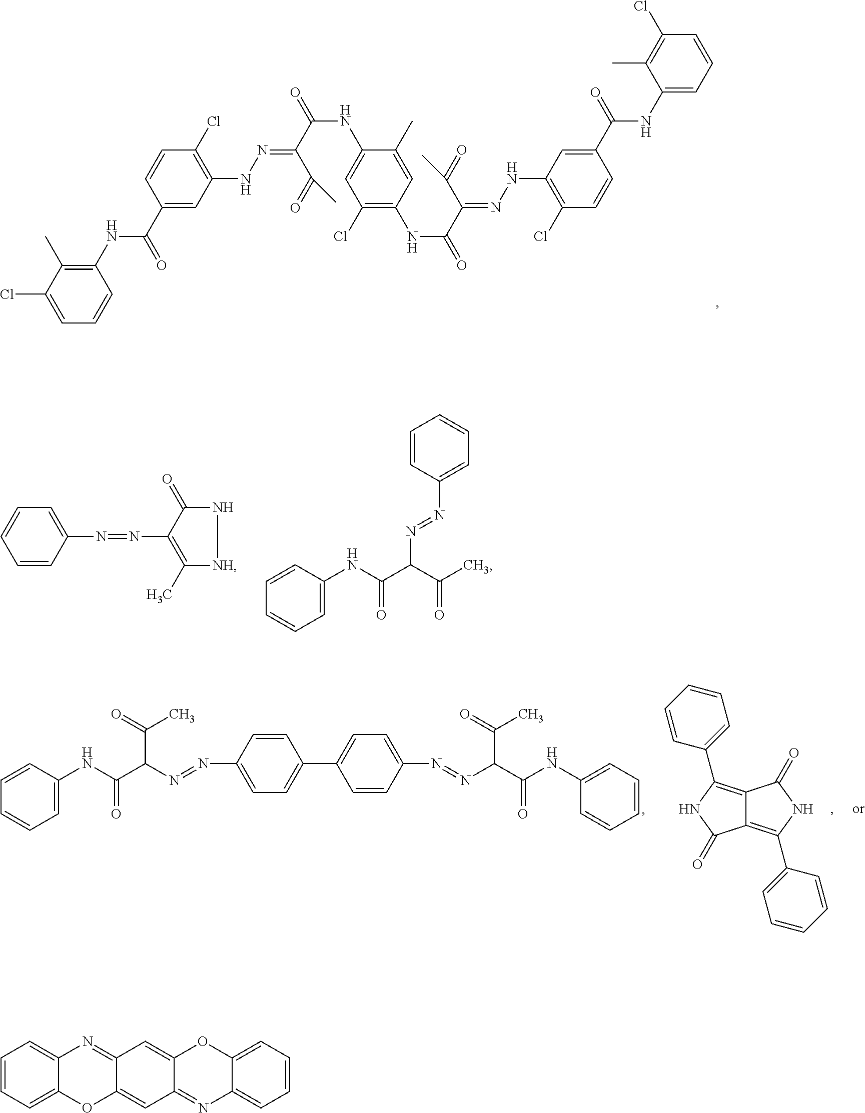

and also, in each case, all known derivatives thereof.

In addition, it is also possible, to use “latent pigments” that are soluble in water/alcohols, preferably in water, for example those that are described in EP-A-1125995. Such water-soluble latent pigments are, for example, latent pigments of formula I wherein B is a group of formula

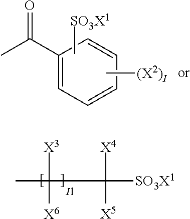

x is a number from 1 to 5, X¹ is a hydrogen atom, an alkali metal cation or an ammonium cation, X² is a substituent, X³, X⁴, X⁵ and X⁶ are a hydrogen atom or a C₁₋₄alkyl radical, I and I1 are a number from 0 to 4, and wherein a plurality of substituents X² may, when 1 is from 2 to 4, be linked to one another to form a ring. Latent pigments of formula I wherein B is a group of formula

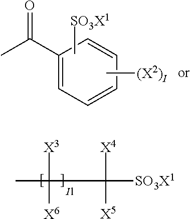

are less preferred.

Worthy of special mention are those soluble chromophores wherein the pigment of formula A(H)ₓ is Colour Index Pigment Yellow 13, Pigment Yellow 73, Pigment Yellow 74, Pigment Yellow 83, Pigment Yellow 93, Pigment Yellow 94, Pigment Yellow 95, Pigment Yellow 109, Pigment Yellow 110, Pigment Yellow 120, Pigment Yellow 128, Pigment Yellow 139, Pigment Yellow 151, Pigment Yellow 154, Pigment Yellow 175, Pigment Yellow 180, Pigment Yellow 181, Pigment Yellow 185, Pigment Yellow 194, Pigment Orange 31, Pigment Orange 71, Pigment Orange 73, Pigment Red 122, Pigment Red 144, Pigment Red 166, Pigment Red 184, Pigment Red 185, Pigment Red 202, Pigment Red 214, Pigment Red 220, Pigment Red 221, Pigment Red 222, Pigment Red 242, Pigment Red 248, Pigment Red 254, Pigment Red 255, Pigment Red 262, Pigment Red 264, Pigment Brown 23, Pigment Brown 41, Pigment Brown 42, Pigment Blue 25, Pigment Blue 26, Pigment Blue 60, Pigment Blue 64, Pigment Violet 19, Pigment Violet 29, Pigment Violet 32, Pigment Violet 37, 3,6-di(4′-cyano-phenyl)-2,5-dihydro-pyrrolo[3,4-c]pyrrole-1,4-dione, 3,6-di(3,4-dichloro-phenyl)-2,5-dihydro-pyrrolo[3,4-c]pyrrole-1,4-dione or 3-phenyl-6-(4′-tert-butyl-phenyl)-2,5-dihydro-pyrrolo[3,4-c]pyrrole-1,4-dione. Further examples are described by Willy Herbst and Klaus Hunger in “Industrial Organic Pigments” (ISBN 3-527-28161-4, VCH/Weinheim 1993). In general, those soluble pigment precursors do not have deprotonatable carboxylic acid or sulfonic acid groups.

Particularly preferred latent pigments are shown below:
a latent pigment based on C.I. Pigment Red 254

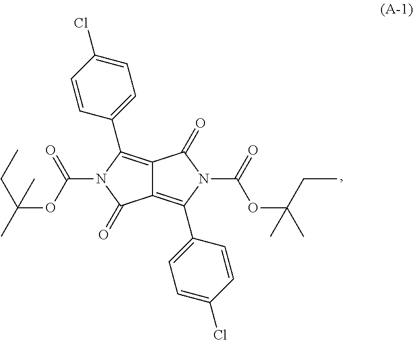

which shows a colour change from fluorescent yellow (latent pigment) to bordeaux red (pigment);
a latent pigment based on C.I. Pigment Red 254

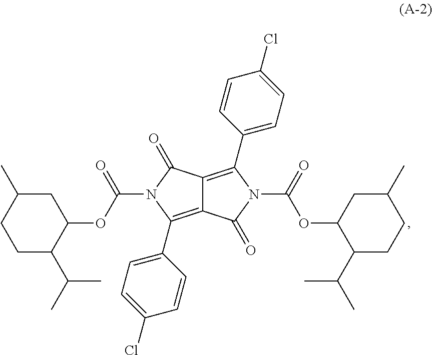

which shows a colour change from fluorescent yellow (latent pigment) to bordeaux red (pigment);
a latent pigment based on C.I. Pigment Violet 37,

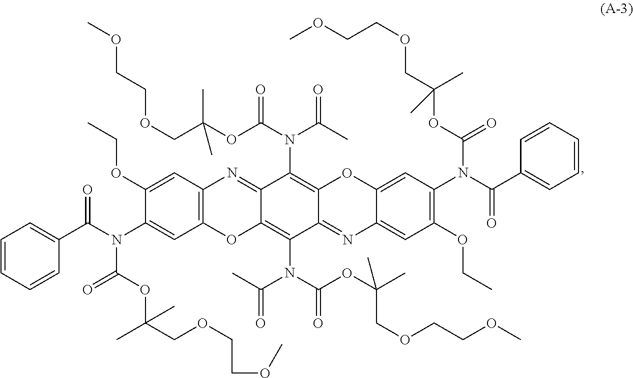

which shows a colour change from magenta (latent pigment) to violet (pigment);
a latent pigment of formula

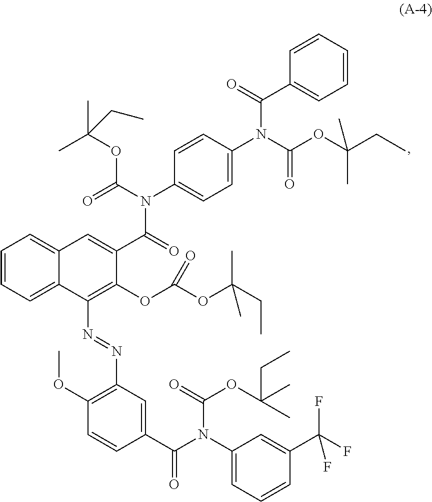

which shows a colour change from fluorescent orange (latent pigment) to magenta (pigment);
a latent pigment of formula

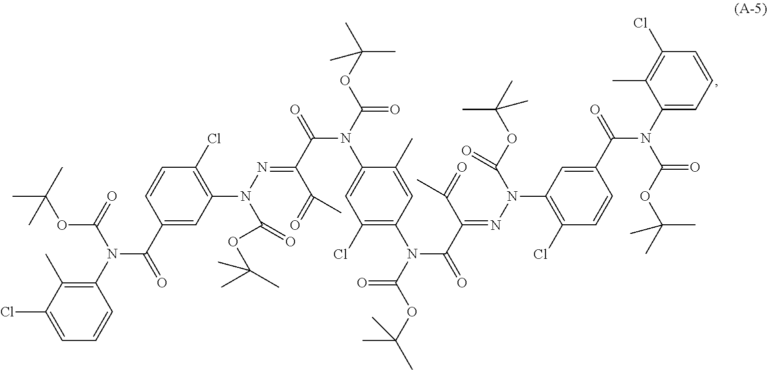

which shows a colour change from colourless ((latent pigment) to yellow (pigment); and
a latent pigment based on of formula

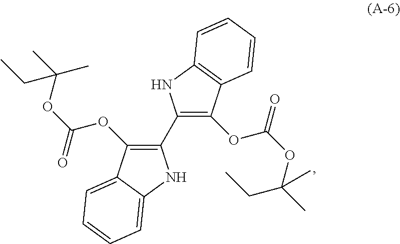

which shows a colour change from colourless in pure form (latent pigment) over violet till blue (pigment). Most preferred is A-1.

Alkyl or alkylene may be straight-chained, branched, monocylic or polycyclic. C₁-C₁₂Alkyl is accordingly, for example, methyl, ethyl, n-propyl, isopropyl, n-butyl, sec-butyl, isobutyl, tert-butyl, cyclobutyl, n-pentyl, 2-pentyl, 3-pentyl, 2,2-dimethylpropyl, cyclopentyl, cyclohexyl, n-hexyl, n-octyl, 1,1,3,3-tetramethylbutyl, 2-ethylhexyl, nonyl, tri methylcyclohexyl, decyl, menthyl, thujyl, bornyl, 1-adamantyl, 2-adamantyl or dodecyl.

When C₂-C₁₂alkyl is mono- or poly-unsaturated, it is C₂-C₁₂alkenyl, C₂-C₁₂alkynyl, C₂-C₁₂alkapolyenyl or C₂-C₁₂alkapolyynyl, it being possible for two or more double bonds to be, where appropriate, isolated or conjugated, such as, for example, vinyl, allyl, 2-propen-2-yl, 2-buten-1-yl, 3-buten-1-yl, 1,3-butadien-2-yl, 2-cyclobuten-1-yl, 2-penten-1-yl, 3-penten-2-yl, 2-methyl-1-buten-3-yl, 2-methyl-3-buten-2-yl, 3-methyl-2-buten-1-yl, 1,4-pentadien-3-yl, 2-cyclopenten-1-yl, 2-cyclohexen-1-yl, 3-cyclohexen-1-yl, 2,4-cyclohexadien-1-yl, 1-p-menthen-8-yl, 4(10)-thujen-10-yl, 2-norbornen-1-yl, 2,5-norbornadien-1-yl, 7,7-dimethyl-2,4-norcaradien-3-yl and the various isomers of hexenyl, octenyl, nonenyl, decenyl and dodecenyl.

C₂-C₄Alkylene is, for example, 1,2-ethylene, 1,2-propylene, 1,3-propylene, 1,2-butylene, 1,3-butylene, 2,3-butylene, 1,4-butylene and 2-methyl-1,2-propylene.

C₅-C₁₂Alkylene is, for example, an isomer of pentylene, hexylene, octylene, decylene or dodecylene.

C₁-C₁₂Alkoxy is O—C₁-C₁₂alkyl, preferably O—C₁-C₄alkyl.

C₆-C₁₂Aryloxy is O—C₆-C₁₂aryl, for example phenoxy or naphthyloxy, preferably phenoxy.

C₁-C₁₂Alkylthio is S—C₁-C₁₂alkyl, preferably S—C₁-C₄alkyl.

C₆-C₂₁Arylthio is S—C₆-C₁₂aryl, for example phenylthio or naphthylthio, preferably phenylthio.

C₂-C₂₄Dialkylamino is N(alkyl₁)(alkyl₂), the sum of the carbon atoms in the two groups alkyl₁ and alkyl₂ being from 2 to 24, preferably N(C₁-C₄alkyl)-C₁-C₄alkyl.

C₇-C₂₄Alkylarylamino is N(alkyl₁)(aryl₂), the sum of the carbon atoms in the two groups alkyl₁ and aryl₂ being from 7 to 24, for example methylphenylamino, ethylnaphthylamino or butylphenanthrylamino, preferably methylphenylamino or ethylphenylamino.

C₁₂-C₂₄Diarylamino is N(aryl₁)(aryl₂), the sum of the carbon atoms in the two groups aryl₁ and aryl₂ being from 12 to 24, for example diphenylamino or phenylnaphthylamino, preferably diphenylamino.

Halogen is chlorine, bromine, fluorine or iodine, preferably fluorine or chlorine, especially chlorine.

Generally, the decrease of the decomposition temperature, or the velocity of decomposition of the latent pigment at a given temperature depends on the acidity (pKₐ-value) and the polarity of the used acid and the nature of the used binder. If the acidity is too high a premature decomposition of the latent pigment may occur at room temperature. If the acidity is too low, no effect may be observed at all. Therefore, the pKₐ-value of the preferred organic acids is in the range of from 1 to 5, especially 1.5 to 4.5. In addition, the preferred acids exhibit a polarity in the medium to low range in the application medium. A vinyl chloride-vinyl acetate copolymer is an example of an application medium.

The rate of acceleration of decomposition is also dependant on the amount of acid used. Generally, an increase of the amount of acid enhances the velocity of the decomposition of the latent pigment at a given temperature. In addition, the temperature which results in a desired decomposition rate may be lowered. Generally, the amount of acid in the printing ink is in the range of 0.1-20% by weight, preferably in the range of 0.5-10% by weight based on the total weight of the printing ink.

The term “organic acid” means an organic compound having a pKₐ-value in the range of from 1 to 5, especially a pKₐ-value in the range of from 1.5 to 4.5. The term also comprises precursors of organic acids, such as, for example, so called “PhotoAcid Generators” (PAGs) or “Thermal Acid Generators” (TAGs). PAGs, or TAGs liberate the desired acid (having the specified pKₐ-value) by means of radiation or application of heat. Suitable PAGs are, for instance, available from Ciba Inc. Examples of such compounds are shown below:

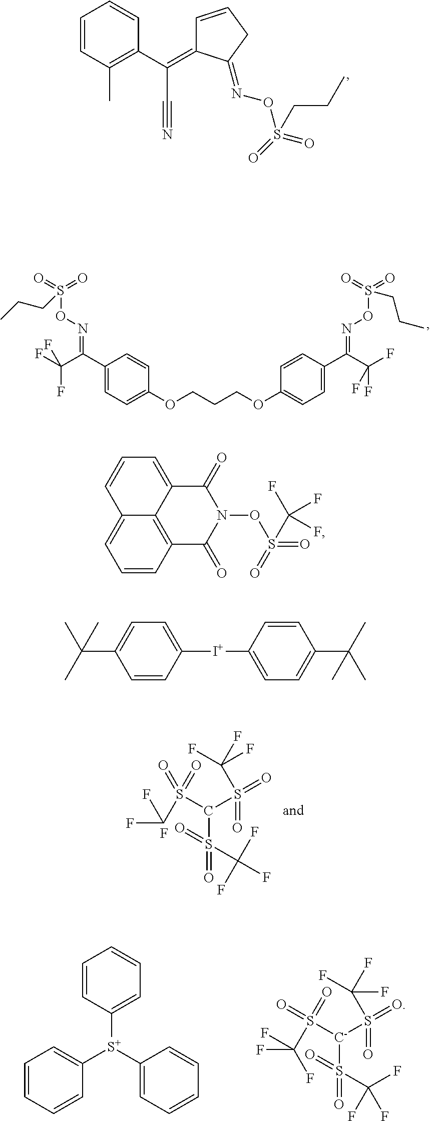

PAGs and TAGs are less preferred.

Examples of organic acids which can be used in the compositions of the present invention are α-carbonyl carboxylic acids, such as, for example, glyoxylic acid and benzoyl formic acid (B-14), benzoic acids, especially substituted benzoic acids, such as, for example, compounds B-1 to B-7, B9 and B11, etc., benzilic acid and derivatives, such as, for example, compound B-8, ascorbic acid and derivatives thereof, such as, for example, esters of ascorbic acid, such as, for example, ascorbylpalmitate (B-15), amino acids, such as, for example, compounds B-16, B-17 and B-18, cyclamate and derivatives thereof, saccharin and derivatives thereof.

By selecting the proper latent pigment as well as the kind and amount of organic acid the indicator system of the present invention can be easily adapted to the desired time-temperature conditions.

In a preferred embodiment of the present invention the organic acid is a compound of formula

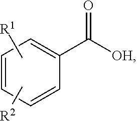

wherein R¹ and R²are independently of each other H, OH, Cl, or NO₂, with the proviso that at least one of R¹ and R²are different from H. The pKₐ-value of said acids is preferably in the range of from 1.5 to 4.5, more preferably in the range of from above 2.0 to 4.0.

Preferred organic acids are the compounds B-1 to B-18 shown in claim 3: 5-chloro-salicylic acid (B-1), 4-chloro-salicylic acid (B-2), 2,4-dihydroxybenzoic acid (B-3), 2-chloro-4-nitro-benzoic acid (B-4), 2-chloro-benzoic acid (B-5), 2,5-dihydroxy-benzoic acid (B-6), salicylic acid (B-7), benzilic acid (B-8), 3-nitro-benzoic acid (B-9), saccharin (B-10), 3,4-dihydroxy-benzoic acid (B-11), cyclamate (B-12), sorbic acid (B-13), phenyl-pyruvic acid (B-14), 6-palmityl-L-ascorbic acid (B-15), L-phenylalanine (B-16), tryptophane (B-17), L-leucine (B-18) and mixtures thereof.

A matrix or medium in which the latent pigments, organic acids and any other additives can be dissolved or dispersed are referred herein to as binders, polymers or polymeric binders. A wide variety of polymeric materials can be used as binders for the indicator formulation as long as the latent pigments and organic acids can be dissolved or dispersed in them. Both aqueous and non-aqueous binders can be used. The binders can be formulated as ink formulations, such as for use as flexo and gravure inks. Other inks such as those for ink jet printing, letter press, offset and screen printing, can also be made and used. Selection of a polymer depends upon the printing/coating equipment to be used.

The formulation of the present invention is preferably an ink. The ink according to the present invention comprises, as in the case of an ordinary printing ink, a latent pigment, an organic acid, a binder, an auxiliary agent, and the like.

With respect to the binder resin, a thermoplastic resin may be used, examples of which include, polyethylene based polymers [polyethylene (PE), ethylene-vinyl acetate copolymer (EVA), vinyl chloride-vinyl acetate copolymer, vinyl alcohol-vinyl acetate copolymer, polypropylene (PP), vinyl based polymers [poly(vinyl chloride) (PVC), poly(vinyl butyral) (PVB), poly(vinyl alcohol) (PVA), poly(vinylidene chloride) (PVdC), poly(vinyl acetate) (PVAc), poly(vinyl formal) (PVF)], polystyrene based polymers [polystyrene (PS), styrene-acrylonitrile copolymer (AS), acrylonitrile-butadiene-styrene copolymer (ABS)], acrylic based polymers [poly(methyl methacrylate) (PMMA), MMA-styrene copolymer], polycarbonate (PC), celluloses [ethyl cellulose (EC), cellulose acetate (CA), propyl cellulose (CP), cellulose acetate butyrate (CAB), cellulose nitrate (CN)], fluorin based polymers [polychlorofluoroethylene (PCTFE), polytetrafluoroethylene (PTFE), tetrafluoroethylene-hexafluoroethylene copolymer (FEP), poly(vinylidene fluoride) (PVdF)], urethane based polymers (PU), nylons [type 6, type 66, type 610, type 11], polyesters (alkyl) [polyethylene terephthalate (PET), polybutylene terephthalate (PBT), polycyclohexane terephthalate (PCT)], novolac type phenolic resins, or the like. In addition, thermosetting resins such as resol type phenolic resin, a urea resin, a melamine resin, a polyurethane resin, an epoxy resin, an unsaturated polyester and the like, and natural resins such as protein, gum, shellac, copal, starch and rosin may also be used.

Further, the above resins may be in an emulsion form for use in a water-based paint. Emulsions for use in a water-based paint include for example, a vinyl acetate (homopolymer) emulsion, a vinyl acetate-acrylic ester copolymer emulsion, a vinyl acetate-ethylene copolymer emulsion (EVA emulsion), a vinyl acetate-vinyl versatate copolymer resin emulsion, a vinyl acetate-polyvinyl alcohol copolymer resin emulsion, a vinyl acetate-vinyl chloride copolymer resin emulsion, an acrylic emulsion, an acryl silicone emulsion, a styrene-acrylate copolymer resin emulsion, a polystyrene emulsion, an urethane polymer emulsion, a polyolefin chloride emulsion, an epoxy-acrylate dispersion, an SBR latex, and the like.

Furthermore, to the binder, a plasticizer for stabilizing the flexibility and strength of the print film and a solvent for adjusting the viscosity and drying property thereof may be added according to the needs therefor. A solvent of a low boiling temperature of about 100° C. and a petroleum solvent of a high boiling temperature of 250° C. or higher, may be used according to the type of the printing method. An alkylbenzene or the like, for example may be used as a solvent of a low boiling temperature. Examples of solvents are ethoxypropanol, methylethylketon, methoxypropylacetate, diacetonalcohol etc.

Further in addition, an auxiliary agent including a variety of reactive agents for improving drying property, viscosity, and dispersibility, may suitably be added. The auxiliary agents are to adjust the performance of the ink, and for example, a compound that improves the abrasion resistance of the ink surface and a drying agent that accelerates the drying of the ink, and the like may be employed.

A photopolymerization-curable resin or an electron beam curable resin wherein a solvent is not used may also be employed as a binder resin that is a principal component of the vehicle. The examples thereof include an acrylic resin, and specific examples of acrylic monomers commercially available are shown below.

A monofunctional acrylate monomer that may be used includes for example, 2-ethylhexyl acrylate, 2-ethylhexyl-EO adduct acrylate, ethoxydiethylene glycol acrylate, 2-hydroxyethyl acrylate, 2-hydroxypropyl acrylate, 2-hydroxyethyl acrylate-caprolactone adduct, 2-phenoxyethyl acrylate, phenoxydiethylene glycol acrylate, nonyl phenol-EO adduct acrylate, (nonyl phenol-EO adduct)-caprolactone adduct acrylate, 2-hydroxy-3-phenoxypropyl acrylate, tetrahydrofurfuryl acrylate, furfuryl alcohol-caprolactone adduct acrylate, acryloyl morpholine, dicyclopentenyl acrylate, dicyclopentanyl acrylate, dicyclopentenyloxyethyl acrylate, isobornyl acrylate, (4,4-dimethyl-1,3-dioxane)-caprolactone adduct acrylate, (3-methyl-5,5-dimethyl-1,3-dioxane)-caprolactone adduct acrylate, and the like.

A polyfunctional acrylate monomer that may be used includes hexanediol diacrylate, neopentyl glycol diacrylate, polyethylene glycol diacrylate, tripropylene glycol diacrylate, neopentyl glycol hydroxypivalate diacrylate, (neopentyl glycol hydroxypivalate)-caprolactone adduct diacrylate, (1,6-hexanediol diglycidyl ether)-acrylic acid adduct, (hydroxypivalaldehyde-trimethylolpropane acetal)diacrylate, 2,2-bis[4-(acryloyloxydiethoxy)phenyl]propane, 2,2-bis[4-(acryloyloxydiethoxy)phenyl]methane, hydrogenated bisphenol A-ethylene oxide adduct diacrylate, tricyclodecanedimethanol diacrylate, trimethylolpropane triacrylate, pentaerithritol triacrylate, (trimethylolpropane-propylene oxide) adduct triacrylate, glycerine-propylene oxide adduct triacrylate, a mixture of dipentaerithritol hexaacrylate and pentaacrylate, esters of dipentaerithritol and lower fatty acid and acrylic acid, dipentaerithritol-caprolactone adduct acrylate, tris(acryloyloxyethyl)isocyanurate, 2-acryloyloxyethyl phosphate, and the like.

Inks comprising the above resins are free of solvent and are so constituted as to polymerize in chain reaction upon irradiation by an electron beam or electromagnetic waves.

With respect to inks of ultraviolet-irradiation type among these inks, a photopolymerization initiator, and depending on the needs therefor, a sensitizing agent, and auxiliary agents such as a polymerization inhibitor and a chain transfer agent, and the like may be added thereto.

With respect to photo-polymerization initiators, there are, (1) an initiator of direct photolysis type including an arylalkyl ketone, an oxime ketone, an acylphosphine oxide, or the like, (2) an initiator of radical polymerization reaction type including a benzophenone derivative, a thioxanthone derivative, or the like, (3) an initiator of cationic polymerization reaction type including an aryl diazonium salt, an aryl iodinium salt, an aryl sulfonium salt, and an aryl acetophenone salt, or the like, and in addition, (4) an initiator of energy transfer type, (5) an initiator of photoredox type, (6) an initiator of electron transfer type, and the like. With respect to the inks of electron beam-curable type, a photopolymerization initiator is not necessary and a resin of the same type as in the case of the ultraviolet-irradiation type inks can be used, and various kinds of auxiliary agent may be added thereto according to the needs therefor.

The inks comprise a total content of latent pigment of from 0.1 to 20% by weight, preferably 0.5-10% by weight, most preferred 1-4% by weight based on the total weight of the ink.

Further, by varying the amount of organic acid in the formulation, the specific conditions at which the indicator composition will change colour can be altered. Such compositions are specifically useful for determining the effectiveness of a sterilization process.

The inks comprise a total content of acid of from 0.1 to 20% by weight, preferably 0.5-10% by weight, most preferred 1-4% by weight based on the total weight of the ink.

The latent pigment-dispersed ink composition can be produced by employing various methods which have so far conventionally been known. For example, it can readily be obtained by blending the respective components (binder, additive, latent pigment and organic acid) and mixing and stirring them by means of a stirrer such as a dissolver or mixing and crushing them by means of a ball mill.

In a preferred embodiment of the present invention the latent pigment is dispersed together with the other respective components using standard dispersing apparatus, such as, for example, a Scandex mixer, or a dissolver (Zahnscheibenrührer). Amounts of latent pigment can vary between 0.5 and 10% by weight, preferably between 1 and 4% by weight based on the total weight of the ink. The acid can be added simultaneously, or in a later stage. Another possibility is the arrangement of the acid in a second layer on top or below the layer containing the latent pigment. The latter technique, which is more time consuming, can be used if the added acid causes a premature (partial) decomposition of the latent pigment in the dispersion/dissolving step.

The indicator can undergo a colour change from a very low temperature (e. g., room temperature) to a very high temperature (e. g., 150° C.) of pressurized steam. The preferred temperature for the colour change depends upon the application of the indicator. For monitoring doneness of a food and sterilization of kitchenware, the temperature could be between 60° C. and 100° C. For monitoring steam sterilization of canned foods the temperature could vary from 80° C. to 120° C. and that of medical supplies it could vary from 100° C. to 150° C. The preferred temperature range is 80 to 140° C.

The time required for the colour change can be varied by varying one or more of the following parameters: thickness of the binder and the indicator layer; concentration of the organic acid; concentration of the latent pigment; and nature of the binder.

The thickness of the indicator and barrier layers may vary from a micron to five hundred microns. The preferred thickness is approximately 0.5-50 microns and the most preferred thickness is approximately 1-20 microns.

The preferred time range for the indicator will depend upon the application and the temperature of sterilization. The preferred time for sterilization is from 1 to 100 minutes. The most preferred time is 2 to 30 minutes. The preferred time range for doneness of food and sterilization of kitchenware also depend upon the temperature of the warm up. The preferred time for doneness of the food is from 1 to 100 minutes. The most preferred time is 2 to 15 minutes.

The devices described here are integrators, i. e., they monitor integral value of time, and temperature.

Medical supplies are usually sterilized above 100° C., e. g., for about 20 minutes at 125° C. and 5 minutes at 135° C. In order to use an indicator as a steam sterilization indicator for medical supplies, the indicator preferably must not undergo the colour change below 100° C. It must also not undergo colour change at high ambient temperature and humidity. The preferred indicator composition for sterilization of medical supplies consists of a 1:1 mixture of A-1 and B-8 in a screen printing formulation (Example 1). Such an indicator composition does not change colour at 40° C. for two weeks.

Frozen foods, to be heated either with microwave oven or convention gas or electric ovens, should preferably be heated above at least 80° C. The time required for doneness of the food will depend upon the nature of the food. The indicator composition to be used for monitoring doneness of food should not change colour below about 60° C.

Homes, restaurants and catering organizations use kitchenware such as dishes, cutlery and utensils, which need to be sterilized with either dry heat, hot water and steam usually below 100° C. There is also a need for an indicator composition, for example, 90° C. for 10 minutes, to make sure the cookware have been subjected to certain integral value of heat and/or humidity.

A composition, comprising a latent pigment, an organic acid having a pKₐ-value in the range of from 1 to 5 is new and forms a further object of the present invention.

Preferred compositions comprise:

| Composition | Latent Pigment | Organic Acid |
|---|---|---|
| C-1 | Cpd. A-1 | Cpd. B-1 |
| C-2 | Cpd. A-1 | Cpd. B-2 |
| C-3 | Cpd. A-1 | Cpd. B-3 |
| C-4 | Cpd. A-1 | Cpd. B-4 |
| C-5 | Cpd. A-1 | Cpd. B-5 |
| C-6 | Cpd. A-1 | Cpd. B-6 |
| C-7 | Cpd. A-1 | Cpd. B-7 |
| C-8 | Cpd. A-1 | Cpd. B-8 |
| C-9 | Cpd. A-1 | Cpd. B-9 |
| C-10 | Cpd. A-1 | Cpd. B-10 |
| C-11 | Cpd. A-1 | Cpd. B11 |
| C-12 | Cpd. A-1 | Cpd. B-12 |
| C-13 | Cpd. A-1 | Cpd. B-13 |
| C-14 | Cpd. A-1 | Cpd. B-14 |
| C-15 | Cpd. A-1 | Cpd. B-15 |
| C-16 | Cpd. A-1 | Cpd. B-16 |
| C-17 | Cpd. A-1 | Cpd. B-17 |
| C-18 | Cpd. A-1 | Cpd. B-18 |
| C-19 | Cpd. A-2 | Cpd. B-3 |
| C-20 | Cpd. A-2 | Cpd. B-9 |
| C-21 | Cpd. A-3 | Cpd. B-7 |
| C-22 | Cpd. A-4 | Cpd. B-3 |
| C-23 | Cpd. A-4 | Cpd. B-7 |
| C-24 | Cpd. A-5 | Cpd. B-3 |
| C-25 | Cpd. A-5 | Cpd. B-7 |
| C-26 | Cpd. A-6 | Cpd. B-7 |
| C-27 | Cpd. A-6 | Cpd. B-8 |

Various features and aspects of the present invention are illustrated further in the examples that follow. While these examples are presented to show one skilled in the art how to operate within the scope of this invention, they are not to serve as a limitation on the scope of the invention where such scope is only defined in the claims. Unless otherwise indicated in the following examples and elsewhere in the specification and claims, all parts and percentages are by weight, temperatures are in degrees centigrade and pressures are at or near atmospheric.

### EXAMPLES

### Example 1

Standard Vinyl Ink Formulation for Screen Printing:

The ink formulation consists of Movital B20H (6.5%, a vinylalcohol/vinylacetate copolymer), Foralyn 5020-F (1.5%), Ethoxypropanol (30%), Methoxypropylacetat (40%), and Diacetonalkohol (22%).

0.5 g latent pigment (for 3%) and 0.5 g of organic acid (for 3%) are mixed together with 16.7 g of the printing ink formulation and 60 g 3 mm glass beads. The mixture is dispersed in a Skandex mixer for 20 minutes and is subsequently applied on black and white paper using a docter blade (#2=layer thickness of 12 μm wet).

Kinetic measurements are performed using a heating plate (Präzitherm 1100, Harry Gestigkeit GmbH Düsseldorf). The colour development is measured every minute using a colorimeter (Eye1, Gretag-Mcbeth).

In table 1 the colour development of compound A-1 with benzilic acid B-8 (1:1; composition C-8) is shown:

| | 100° C. | | | 125° C. | | | 135° C. | | |
|---|---|---|---|---|---|---|---|---|---|
| Time (min.) | L | a | b | L | a | b | L | a | b |
| 0 | 97.50 | −21.38 | 62.38 | 97.14 | −21.80 | 66.55 | 97.19 | −21.54 | 62.81 |
| 1 | 97.29 | −19.98 | 63.58 | 87.22 | 5.83 | 64.63 | 80.22 | 21.65 | 44.25 |
| 2 | 96.63 | −16.54 | 65.13 | 81.89 | 17.89 | 47.77 | 72.26 | 37.63 | 34.47 |
| 3 | 96.17 | −14.01 | 63.41 | 77.07 | 27.86 | 41.22 | 68.44 | 44.71 | 29.66 |
| 4 | 94.76 | −10.62 | 64.60 | 74.61 | 32.96 | 37.10 | 65.24 | 49.87 | 26.33 |
| 5 | 94.34 | −9.40 | 59.55 | 72.16 | 37.65 | 33.74 | 63.61 | 52.42 | 24.80 |
| 6 | 94.24 | −9.08 | 58.98 | 66.96 | 47.51 | 30.87 | 62.48 | 53.91 | 24.46 |
| 7 | 93.94 | −8.24 | 57.92 | 68.50 | 44.39 | 30.62 | 61.92 | 54.63 | 23.33 |
| 8 | 90.78 | −1.37 | 56.73 | 68.45 | 44.40 | 29.27 | 61.81 | 54.63 | 22.43 |
| 9 | 90.92 | −1.05 | 53.10 | 65.58 | 50.25 | 25.76 | 60.95 | 55.82 | 22.86 |
| 10 | 89.01 | 2.59 | 52.38 | 66.33 | 47.92 | 28.28 | 60.88 | 55.80 | 22.33 |
| 11 | 89.96 | 0.24 | 53.33 | 65.38 | 50.62 | 23.95 | 61.00 | 55.55 | 21.88 |
| 12 | 88.21 | 5.17 | 47.87 | 64.95 | 49.92 | 27.38 | 60.83 | 55.81 | 21.80 |

### Example 2

Example 1 is repeated, except, that phenyl-pyruvic acid (B-14) is used as organic acid instead of benzilic acid (composition C-14).

In table 2 the colour development of a mixture of compound A-1 with phenyl-pyruvic acid (B-14) (1:1) is shown (composition C-14):

| | 100° C. | | | 125° C. | | | 135° C. | | |
|---|---|---|---|---|---|---|---|---|---|
| Time (min.) | L | a | b | L | a | b | L | a | b |
| 0 | 96.6 | −20.05 | 65.1 | 96.29 | −20.86 | 73.80 | 96.37 | −20.67 | 69.91 |
| 1 | 96.48 | −20.33 | 66.63 | 92.49 | −8.33 | 69.34 | 85.38 | 8.76 | 58.69 |
| 2 | 96.45 | −19.54 | 66.35 | 86.84 | 5.37 | 61.67 | 77.15 | 26.46 | 45.17 |
| 3 | 96.39 | −18.69 | 65.39 | 81.69 | 16.40 | 54.72 | 72.06 | 36.64 | 39.14 |
| 4 | 96.20 | −17.52 | 64.58 | 78.70 | 22.97 | 49.48 | 68.50 | 43.14 | 34.27 |
| 5 | 95.88 | −16.81 | 64.96 | 76.21 | 27.97 | 45.45 | 65.54 | 48.28 | 29.97 |
| 6 | 95.48 | −15.90 | 64.94 | 73.87 | 32.53 | 43.05 | 65.20 | 49.05 | 29.01 |
| 7 | 95.40 | −14.75 | 63.90 | 73.60 | 32.91 | 39.29 | 64.02 | 50.96 | 27.71 |
| 8 | 95.19 | −13.96 | 63.17 | 70.84 | 38.38 | 38.01 | 62.91 | 52.83 | 25.41 |
| 9 | 94.65 | −12.79 | 63.58 | 69.29 | 41.02 | 37.45 | 63.00 | 52.18 | 25.35 |
| 10 | 94.27 | −11.92 | 62.99 | 68.36 | 42.70 | 35.90 | 62.38 | 53.53 | 24.18 |
| 11 | 93.83 | −10.52 | 61.72 | 67.62 | 43.87 | 35.05 | 61.97 | 54.06 | 23.49 |
| 12 | 93.55 | −9.26 | 61.69 | 66.60 | 45.93 | 33.40 | 60.85 | 55.55 | 22.28 |

### Example 3

Example 1 is repeated, except, that saccharin (B-10) is used as organic acid instead of benzilic acid. In table 3 the colour development of compound A-1 with saccharin (B-10) (1:1; composition (C-10)) is shown:

| | 100° C. | | | 125° C. | | | 135° C. | | |
|---|---|---|---|---|---|---|---|---|---|
| Time (min.) | L | a | b | L | a | b | L | a | b |
| 0 | 97.61 | −24.66 | 80.43 | 97.85 | −24.76 | 78.26 | 97.6 | −24.3 | 75.84 |
| 1 | 96.75 | −19.10 | 79.95 | 82.05 | 17.88 | 52.11 | 76.94 | 27.86 | 41.65 |
| 2 | 94.07 | −12.18 | 80.16 | 76.16 | 30.61 | 40.41 | 70.21 | 41.22 | 29.88 |
| 3 | 92.34 | −7.17 | 72.4 | 73.49 | 36.01 | 35.44 | 68.52 | 44.33 | 27.28 |
| 4 | 90.08 | −1.45 | 68.49 | 70.69 | 41.22 | 31.23 | 66.44 | 47.6 | 27.41 |
| 5 | 87.64 | 3.62 | 66.66 | 69.29 | 43.56 | 29.45 | 65.73 | 48.72 | 25.75 |
| 6 | 87.41 | 5.17 | 60.60 | 68.41 | 44.74 | 28.56 | 63.96 | 51.15 | 25.99 |
| 7 | 85.14 | 10.08 | 57.77 | 67.73 | 45.92 | 27.73 | 65.46 | 46.99 | 19.06 |
| 8 | 83.68 | 12.39 | 60.87 | 67.24 | 46.53 | 27.00 | 61.88 | 53.47 | 25.69 |
| 9 | 82.62 | 14.87 | 59.18 | 65.96 | 48.40 | 26.07 | 61.88 | 53.84 | 24.42 |
| 10 | 83.61 | 14.16 | 51.83 | 65.6 | 49.02 | 25.85 | 61.53 | 54.25 | 23.38 |
| 11 | 81.27 | 18.58 | 50.77 | 64.53 | 50.57 | 25.29 | 60.88 | 54.75 | 22.80 |
| 12 | 80.32 | 20.02 | 53.84 | 64.52 | 50.46 | 25.08 | 61.14 | 54.7 | 22.74 |

### Example 4

Example 1 is repeated, except that 4-chloro salicylic acid (B-2) is used as organic acid instead of benzilic acid and the amount of organic acid is varied:

The results are represented in FIGS. 1 and 2. As evident from FIG. 1 the decomposition temperature of the latent pigment is dependant on the amount of acid which is used. If more acid is used, the decomposition temperature is lower.

### Example 5

Standard Vinyl Ink Formulation for Gravure Printing:

The ink formulation consists of Vinylite VYHH (14%, a vinylacetate/vinylchloride copolymer), Ethoxypropanol (10%), Methylethylketon (76%).

0.5 g latent pigment (for 3%) and 0.5 g of organic acid (for 3%) are mixed together with 16.7 g of the printing ink formulation and 60 g 3 mm glass beads. The mixture is dispersed in a Skandex mixer for 20 minutes and is subsequently applied on black and white paper using a docter blade (#2=layer thickness of 12 μm wet).

Kinetic measurements are performed using a heating plate (Präzitherm 1100, Harry Gestigkeit GmbH Düsseldorf). The colour development is measured every minute using a colorimeter (Eye1, Gretag-Mcbeth).

In table 4 the colour development of compound A-1 with benzilic acid (B-8) (1:1; composition C-8) is shown:

| | 80° C. | | | 100° C. | | | 135° C. | | |
|---|---|---|---|---|---|---|---|---|---|
| Time (min) | L | a | b | L | a | b | L | a | b |
| 0 | 97.12 | −22.04 | 105.84 | 96.47 | −20.59 | 110.6 | 96.45 | −20.78 | 107.46 |
| 1 | 97.11 | −21.49 | 106.45 | 95.92 | −18.30 | 111.7 | 69.14 | 41.40 | 60.45 |
| 2 | 96.89 | −20.49 | 106.18 | 93.39 | −11.0 | 110.18 | 63.66 | 52.54 | 40.84 |
| 3 | 96.57 | −19.71 | 105.61 | 90.64 | −4.44 | 106.46 | 63.01 | 53.92 | 38.59 |
| 4 | 96.85 | −18.33 | 107.07 | 88.55 | 1.26 | 102.97 | 61.34 | 57.16 | 32.37 |
| 5 | 95.81 | −17.09 | 105.86 | 83.58 | 11.73 | 98.38 | 59.15 | 60.56 | 27.61 |
| 6 | 96.05 | −16.35 | 106.29 | 82.15 | 14.61 | 93.31 | 59.5 | 60.31 | 26.68 |
| 7 | 95.22 | −15.11 | 105.30 | 79.74 | 21.20 | 91.53 | 58.22 | 61.88 | 26.70 |
| 8 | 94.77 | −14.21 | 105.21 | 77.95 | 23.59 | 88.92 | 58.78 | 61.28 | 25.49 |
| 9 | 94.56 | −13.41 | 104.67 | 76.88 | 25.66 | 86.58 | 58.14 | 61.74 | 25.03 |
| 10 | 94.37 | −12.52 | 104.22 | 75.58 | 28.95 | 84.06 | 57.53 | 62.76 | 25.66 |
| 11 | 94.32 | −11.96 | 103.13 | 74.15 | 31.26 | 81.27 | 57.69 | 62.59 | 25.41 |
| 12 | 93.43 | −11.04 | 103.03 | 73.19 | 33.37 | 78.53 | 57.33 | 62.96 | 25.39 |
| 13 | 93.66 | −10.32 | 103.47 | 72.25 | 35.27 | 76.52 | 57.18 | 63.11 | 25.26 |
| 14 | 92.74 | −9.34 | 102.29 | 71.24 | 36.74 | 74.47 | 57.06 | 63.20 | 25.2 |
| 15 | 92.80 | −8.06 | 103.34 | 69.95 | 37.96 | 71.29 | 57.07 | 63.22 | 25.06 |
| 16 | 92.24 | −7.90 | 101.14 | 69.24 | 39.27 | 70.12 | 56.89 | 63.38 | 25.07 |
| 17 | 91.73 | −6.68 | 101.32 | 68.55 | 40.65 | 67.67 | 57.67 | 62.38 | 24.00 |
| 18 | 92.26 | −6.30 | 101.63 | 68.25 | 41.14 | 67.50 | 57.56 | 62.63 | 24.01 |
| 19 | 91.34 | −6.03 | 99.77 | 67.72 | 42.36 | 65.17 | 57.58 | 62.47 | 23.83 |
| 20 | 91.00 | −4.93 | 99.82 | 67.44 | 42.72 | 63.85 | 57.22 | 62.87 | 24.11 |

### Example 6

Example 5 is repeated, except, that 6-palmityl-L-ascorbinic acid (B-15) is used as organic acid instead of benzilic acid. In table 5 the colour development of compound A-1 with 6-palmityl-L-ascorbinic acid (B-15) (1:1; composition C-15) is shown:

| | 80° C. | | | 100° C. | | | 135° C. | | |
|---|---|---|---|---|---|---|---|---|---|
| Time (min) | L | a | b | L | a | b | L | a | b |
| 0 | 93.49 | −20.35 | 104.85 | 94.97 | −17.49 | 110.97 | 95.22 | −19.11 | 103.14 |
| 1 | 95.14 | −18.52 | 108.50 | 92.95 | −12.93 | 113.40 | 81.32 | 15.69 | 82.89 |
| 2 | 95.29 | −17.77 | 108.93 | 92.26 | −11.16 | 111.55 | 71.70 | 35.88 | 65.54 |
| 3 | 95.30 | −17.49 | 109.49 | 91.50 | −8.04 | 111.27 | 68.07 | 42.85 | 54.12 |
| 4 | 94.92 | −16.93 | 108.94 | 91.34 | −7.30 | 108.56 | 66.42 | 46.35 | 43.87 |
| 5 | 94.43 | −16.42 | 108.10 | 90.54 | −4.68 | 107.08 | 64.46 | 49.91 | 38.33 |
| 6 | 94.15 | −15.56 | 108.62 | 90.24 | −4.51 | 104.06 | 64.00 | 50.81 | 33.78 |
| 7 | 94.05 | −15.22 | 108.18 | 89.71 | −2.72 | 102.78 | 63.26 | 52.23 | 31.43 |
| 8 | 93.88 | −15.11 | 107.51 | 86.56 | 3.05 | 102.73 | 61.87 | 54.25 | 30.57 |
| 9 | 93.77 | −14.50 | 108.08 | 87.58 | 0.61 | 99.08 | 61.44 | 55.03 | 29.13 |
| 10 | 93.58 | −14.93 | 106.08 | 87.03 | 1.94 | 97.80 | 61.91 | 54.26 | 27.66 |
| 11 | 93.35 | −13.44 | 107.80 | 87.21 | 3.09 | 97.28 | 60.92 | 55.89 | 27.78 |
| 12 | 93.76 | −13.51 | 106.92 | 84.85 | 7.24 | 95.95 | 61.40 | 55.11 | 26.82 |
| 13 | 93.03 | −13.46 | 105.89 | 85.54 | 5.63 | 93.98 | 60.49 | 56.36 | 27.20 |
| 14 | 92.53 | −14.08 | 103.87 | 84.80 | 7.02 | 93.10 | 60.28 | 56.64 | 26.89 |
| 15 | 92.74 | −13.11 | 104.60 | 83.50 | 8.35 | 91.19 | 60.64 | 56.10 | 26.29 |
| 16 | 92.86 | −12.58 | 105.61 | 82.29 | 10.89 | 89.76 | 60.18 | 56.61 | 26.33 |
| 17 | 92.66 | −12.30 | 105.59 | 82.45 | 10.69 | 88.92 | 60.87 | 55.79 | 25.42 |
| 18 | 92.60 | −11.94 | 105.67 | 81.77 | 12.03 | 87.38 | 60.30 | 56.33 | 25.63 |
| 19 | 92.50 | −11.50 | 105.52 | 81.16 | 13.61 | 86.18 | 60.02 | 56.85 | 25.75 |
| 20 | 92.13 | −11.92 | 103.63 | 80.84 | 13.94 | 85.40 | 59.71 | 57.35 | 25.72 |

1. An indicator formulation, comprising:
a latent pigment,
an organic acid having a pKₐ-value of from 1.5 to 4.5, and
a binder,
wherein the latent pigment comprises a group that makes the latent pigment soluble in an application medium and that can be split off by heater radiation, which converts the latent pigment to a pigment form.

2. A device, comprising:
(A) at least one layer of polymer comprising a latent pigment capable of undergoing at least one color change and an organic acid; or
(B) one layer of polymer comprising a latent pigment capable of undergoing at least one color change, and one layer of polymer comprising an organic acid,
wherein conversion of the latent pigment to a pigmentary form causes the color change,
the latent pigment comprises a group that makes the latent pigment soluble in an application medium and that can be split off by heat or radiation, which converts the latent pigment to a pigment form, and
a pKₐ-value of the organic acid is from 1 to 5.

3. The indicator formulation of claim 1, wherein the organic acid is a compound of formula 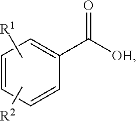
wherein
R¹ and R² are each independently H, OH, Cl, or NO₂, and
at least one of R¹ and R² is not H.

4. The indicator formulation of claim 1, wherein the organic acid is selected from the group consisting of 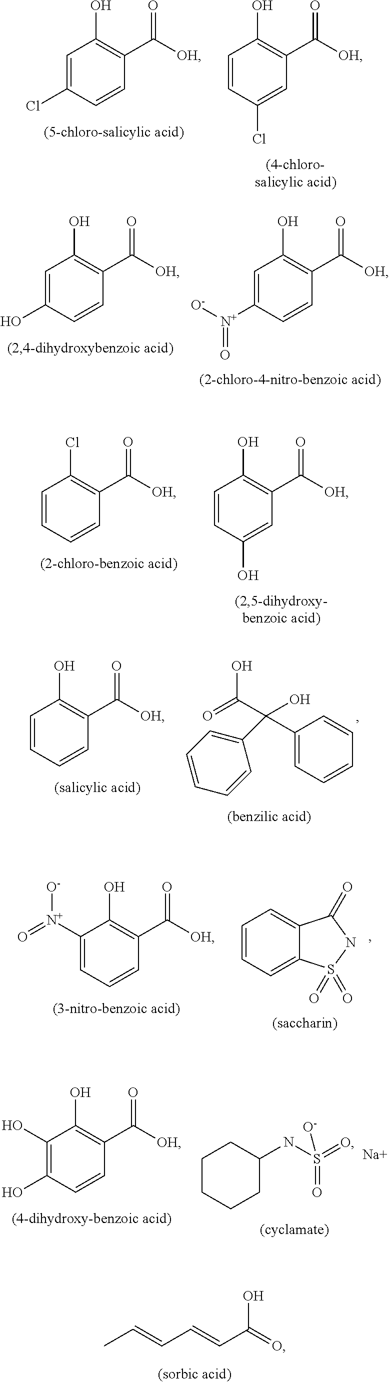 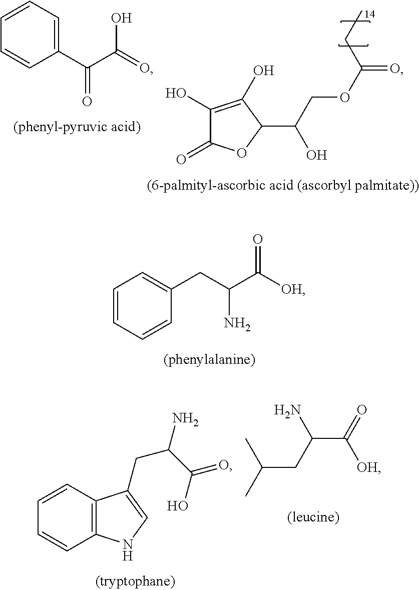
and mixtures thereof.

5. The indicator formulation of claim 1, wherein the latent pigment is of formula A(B)ₓ, wherein
A is a radical of a chromophore of a quinacridone, anthraquinone, perylene, indigo, quinophthalone, indanthrone, isoindolinone, isoindoline, dioxazine, azo, phthalocyanine or diketopyrrolopyrrole series, which is linked to x groups B by one or more hetero atoms of the radical A, the hetero atoms selected from the group consisting of nitrogen, oxygen, and sulfur; and
each B is independently a group of the formula 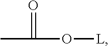
wherein L is any desired group suitable for imparting solubility, and x is an integer from 1 to 8,
B is a group of formula 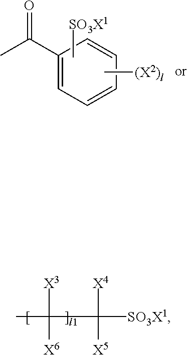
x is an integer from 1 to 5,
X¹ is a hydrogen atom, an alkali metal cation or an ammonium cation,
X² is a substituent,
X³, X⁴, X⁵ and X⁶ are each independently a hydrogen atom or a C₁-C₄ alkyl radical,
1 and I1 are each independently a number from 0 to 4, and
when 1 is from 2 to 4, a plurality of substituents X² are optionally linked to one another to form a ring.

6. The indicator formulation of claim 5, wherein the latent pigment is selected from the group consisting of 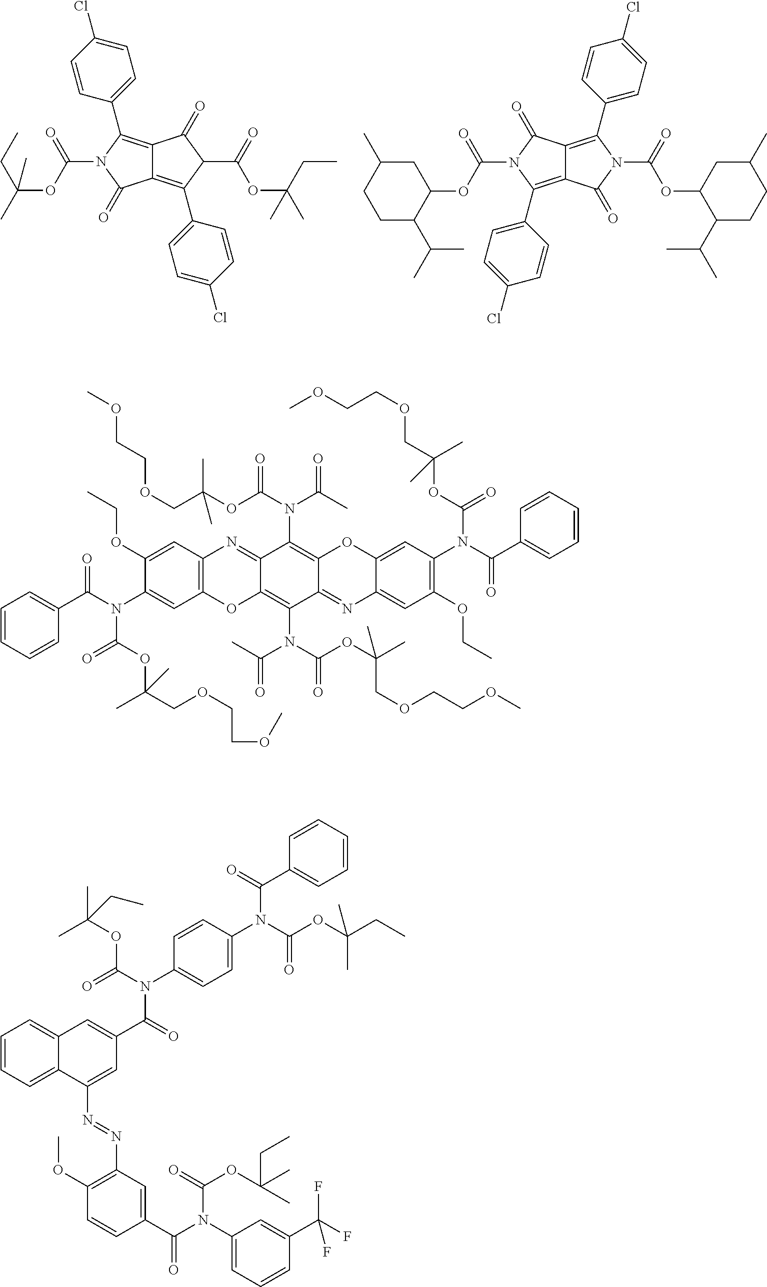 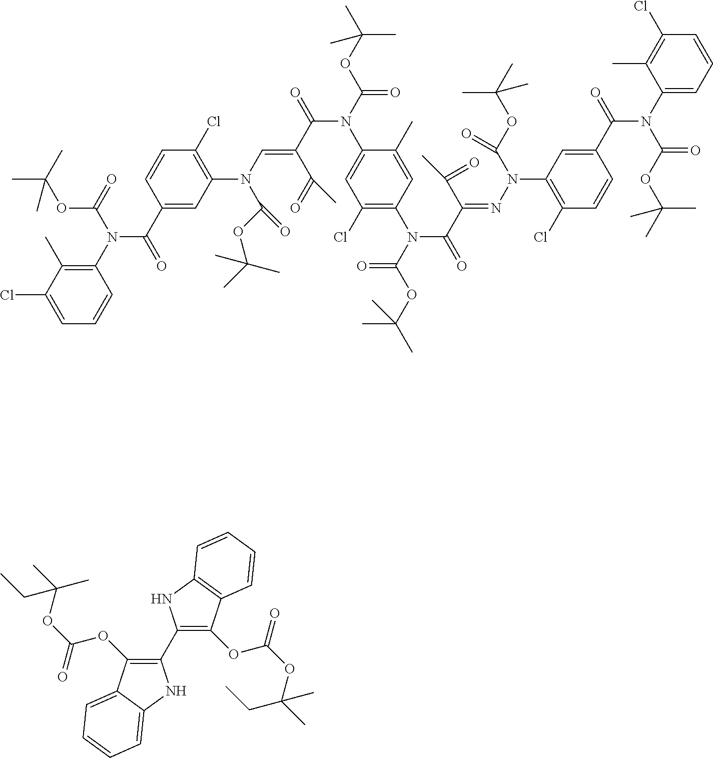
and mixtures thereof.

7. A printing ink, comprising the indicator formulation of claim 1.

8. The printing ink of claim 7,
wherein the binder is selected from the group consisting of
a thermoplastic resin,
a natural resin,
a photopolymerization-curable resin, and
an electron beam curable resin.

9. A process of making the device of claim 2, comprising:
applying an indicator formulation to a substrate,
wherein the indicator formulation comprises:
the latent pigment,
an organic acid having a pKₐ-value of from 1.5 to 4.5, and
a binder.

10. A process for monitoring sterilization of a material, comprising:
affixing the device of claim 2 to the material or to a container containing the material;
sterilizing the material;
heating during the sterilizing; and
observing a color change indicating sterilization.

11. The process of claim 9, wherein the substrate comprises a polymeric material, paper, a ceramic material, a metal, a woven fiber, a non-woven fiber, or a combination thereof.

12. The process of claim 11, wherein the substrate comprises a polymeric material comprising polyethylene, polypropylene, polyvinyl chloride, polymethylmethacrylate, a polyurethane, nylon, polyester, a polycarbonate, a polyvinyl acetate, cellophane, an ester of cellulose, or a mixture thereof.

13. The process of claim 10, wherein the material is a medical supply, a food, a pharmaceutical, or a biological waste.

14. A process for monitoring temperature, comprising:
exposing the device of claim 2 to heat, and
observing a color change in the device.

15. A composition, comprising:
a latent pigment, and
an organic acid having a pKₐ-value of from 1.5 to 4.5,
wherein the latent pigment comprises a group that makes the latent pigment soluble in an application medium and that can be split off by heat or radiation, which converts the latent pigment to a pigment form.

16. The indicator formulation of claim 5, wherein
each B is independently a group of the formula 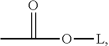
L is a group of formula 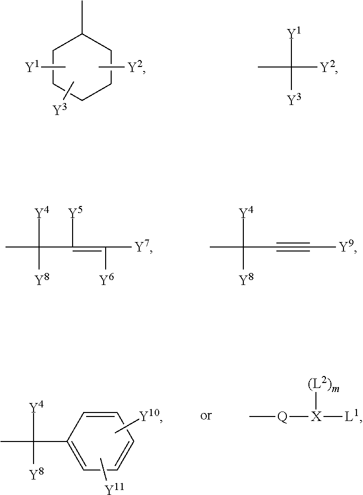
wherein Y¹, Y², and Y³ are each independently a C₁-C₆alkyl,
Y⁴ and Y⁸ are each independently C₁-C₆alkyl; C₁-C₆alkyl interrupted by oxygen, sulphur, or N(Y¹²)₂; or unsubstituted or C₁-C₆alkyl-, C₁-C₆alkoxy-, halo-, cyano- or nitro-substituted phenyl or biphenyl,
Y⁵, Y⁶, and Y⁷ are each independently of the others hydrogen or C₁-C₆alkyl,
Y⁹ is hydrogen, C₁-C₆alkyl or a group of formula 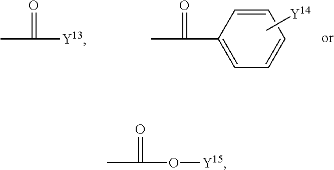
Y¹⁰ and Y¹¹ are each independently hydrogen, C₁-C₆alkyl; C₁-C₆alkoxy; halogen; cyano; nitro; N(Y¹²)₂; or unsubstituted or halo-, cyano-, nitro-, C₁-C₆alkyl- or C₁-C₆alkoxy-substituted phenyl,
Y¹² and Y¹³ are C₁-C₆alkyl,
Y¹⁴ is hydrogen or C₁-C₆alkyl,
Y¹⁵ is hydrogen, C₁-C₆alkyl, or unsubstituted or C₁-C₆alkyl-substituted phenyl,
Q is p,q-C₂-C₆alkylene, unsubstituted or mono- or poly-substituted by C₁-C₆alkoxy, C₁-C₆alkylthio, or C₂-C₁₂dialkylamino, wherein p and q are different position numbers,
X is a hetero atom selected from the group consisting of nitrogen, oxygen, and sulfur,
m is 0 when X is oxygen or sulphur, or m is 1 when X is nitrogen,
L¹ and L² are each independently unsubstituted or mono- or poly-C₁-C₁₂alkoxy-, —C₁-C₁₂alkylthio-, —C₂-C₂₄dialkylamino-, —C₆-C₁₂aryloxy-, —C₆-C₁₂arylthio-, —C₇-C₂₄alkylarylamino- or —C₁₂-C₂₄diarylamino-substituted C₁-C₆alkyl or [-(p′,q′-C₂-C₆alkylene)-Z—]ₙ—C₁-C₆alkyl,
n is an integer from 1 to 1000,
p′ and q′ are each position numbers,
each Z is independently a hetero atom oxygen, sulfur or C₁-C₁₂alkyl-substituted nitrogen,
each C₂-C₆alkylene in [—C₂-C₆alkyleneZ—] can be the same as or different from any other C₂-C₆alkylene, and
L₁ and L₂ may be saturated or unsaturated from one to ten times, is optionally uninterrupted or interrupted at any location by from 1 to 10 groups selected from the group consisting of —(C═O)— and —C₆H₄—, and and optionally comprises from 1 to 10 further substituents selected from the group consisting of halogen, cyano, and nitro substituents.

17. The device of claim 2, wherein the organic acid is a compound of formula 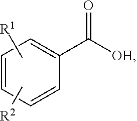
wherein
R¹ and R² are each independently H, OH, Cl, or NO₂, and
at least one of R′ and R² is not H.

18. The device of claim 2, wherein the organic acid is selected from the group consisting of 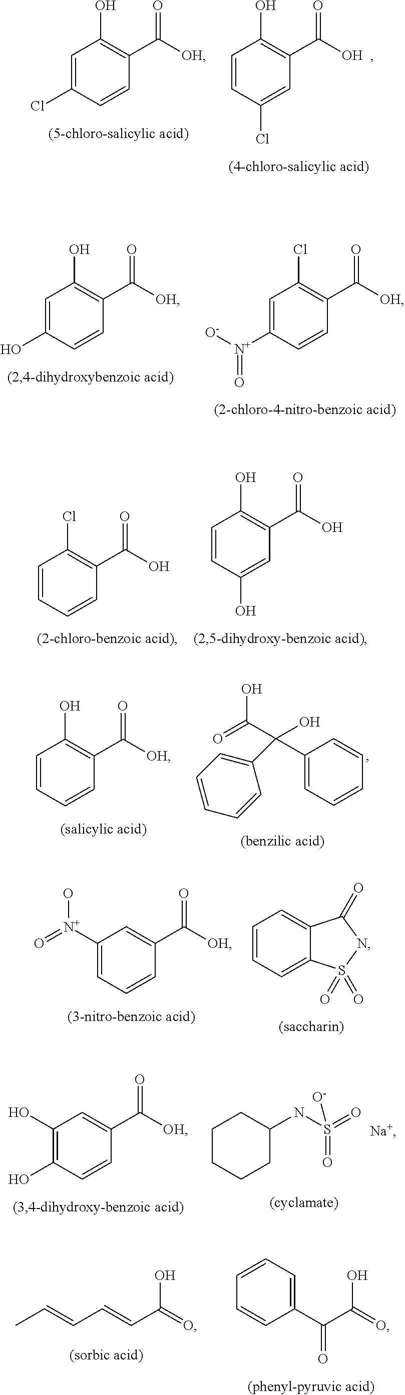 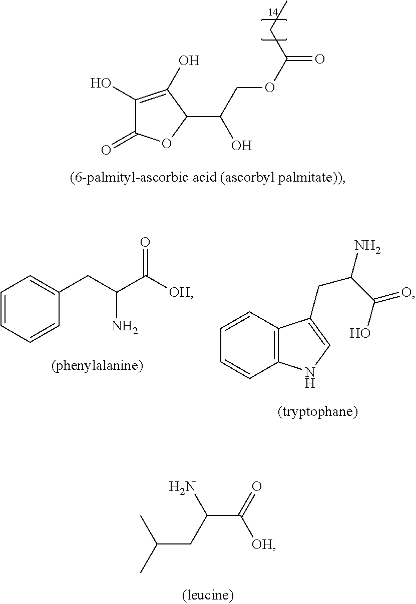
and mixtures thereof.

19. The device of claim 2, wherein the latent pigment is of formula A(B)ₓ, wherein
A is a radical of a chromophore of a quinacridone, anthraquinone, perylene, indigo, quinophthalone, indanthrone, isoindolinone, isoindoline, dioxazine, azo, phthalocyanine or diketopyrrolopyrrole series, which is linked to x groups B by one or more hetero atoms of the radical A, the hetero atoms selected from the group consisting of nitrogen, oxygen, and sulfur; and
each B is independently a group of the formula 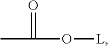
wherein L is any desired group suitable for imparting solubility, and x is an integer from 1 to 8, or
B is a group of formula 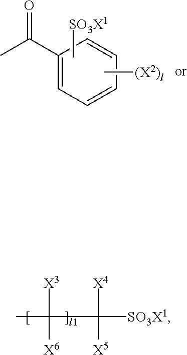
x is an integer from 1 to 5,
X¹ is a hydrogen atom, an alkali metal cation, or an ammonium cation,
X² is a substituent,
X³, X⁴, X⁵ and X⁶ are each independently a hydrogen atom or a C₁-C₄alkyl radical, 1 and I1 are each independently a number from 0 to 4, and
a plurality of substituents X² may, when 1 is from 2 to 4, be linked to one another to form a ring.

20. The printing ink of claim 8,
wherein the binder is a thermoplastic resin selected from the group consisting of a polyethylene based polymer, a polypropylene (PP), a vinyl based polymer, a polystyrene based polymer, an acrylic based polymer, a polycarbonate (PC), a cellulose, a fluorin based polymer, a urethane based polymer (PU), a nylon, a polyester (alkyl), and a novolac type phenolic resin, or
wherein the binder is a thermosetting resin selected from the group consisting of a resol type phenolic resin, a urea resin, a melamine resin, a polyurethane resin, an epoxy resin, and an unsaturated polyester, or
wherein the binder is a natural resin selected from the group consisting of protein, gum, shellac, copal, starch and rosin.
